(19) United States
(12) Patent Application Publication
(10) Pub. No.: US 2014/0121511 A1
(43) Pub. Date: May 1, 2014

(54) Rapid Stress-Rest Cardiac PET Imaging Systems and Methods

(71) Applicant: The University of Utah Research Foundation, (US)

(72) Inventors: Dan J. Kadrmas, North Salt Lake, UT (US); Thomas C. Rust, Salt Lake City, UT (US)

(73) Assignees: The University of Utah; The University of Utah Research Foundation, Salt Lake City, UT (US)

(21) Appl. No.: 13/664,161

(22) Filed: Oct. 30, 2012

(51) Int. Cl. A61B 6/00

(52) U.S. Cl. CPC A61B 6/481; A61B 6/482
USPC 600/431

(57) ABSTRACT

Systems and methods which utilize stress first and then rest imaging techniques to provide for improved medical imaging results are provided herein. One embodiment may administer a stress regime, such as exercise stress, and administer a tracer to a patient and retrieve a stress image. Then a second tracer is administered to a patient and a resting image is retrieved. Embodiments may implement this method with PET/CT scanning techniques. Additionally, embodiments may utilize a single CT scan when obtaining both the stress and rest images.

### TECHNICAL FIELD

The present invention relates to the art of diagnostic imaging. In particular, it relates to positron emission tomography (PET) and other diagnostic modes in which a subject is examined and an image of the subject is reconstructed from information obtained during the examination.

### BACKGROUND

Previously, PET has been used to study a radionuclide distribution in subjects. Typically, one or more radiopharmaceuticals (i.e., tracers) are injected into a subject. The radiopharmaceuticals are commonly injected into the subject's blood stream for imaging the circulatory system or for imaging specific organs which absorb the injected radiopharmaceuticals. PET is a physiologic imaging modality that images the distribution of radiolabeled tracers within the body. Unlike anatomic imaging modalities, which image tissue structures and morphology, PET can characterize the functional, metabolic, and physiologic status of tissues in vivo. Hundreds, if not thousands, of radiotracers have been investigated for PET, targeting parameters such as glucose metabolism, blood flow, hypoxia, cellular proliferation, amino acid synthesis, gene expression, and so on. As more is learned about the molecular bases for disease and treatment, PET becomes an increasingly powerful modality for characterizing and monitoring disease.

In some instances a subject must undergo multiple injections of tracers and scans associated with each tracer. Subsequent injections may be the same tracer as the first, or they may each be a different tracer. Prior to each injection, sufficient time must elapse to allow the earlier introduced tracer to flush from the subject or to decay. This decreases throughput of patients and is inconvenient for patients in clinical applications. To alleviate some of these challenges, rapid multi-tracer PET has been investigated. For instance, rate parameters for individual tracers have been recovered from data with overlapping signals from different PET tracers based on different half-lives, tracer kinetics, or both (Huang et al. 1982; Koeppe et al 1998 and 2001; Converse et al. 2004 and Kadrmas and Rust 2005). In 1982, Huang et al. demonstrated in a phantom that, when imaging static distributions of multiple PET tracers with different half-lives, images of each tracer can be recovered based on their different rates of radioactive decay. In short, this amounts to treating the dynamic PET signal as a sum of exponentials with known decay constants and estimating the coefficients of each exponential. While an important contribution, this approach has little or no practical application because (i) PET tracers are rarely static, except for irreversible tracers long after injection; and (ii) separation of summed exponentials is a poorly conditioned problem sensitive to statistical noise—requiring long scan durations relative to the half-lives of the tracers used in order to get acceptable results. In 1998, Koeppe et al. recovered kinetic rate parameters for two ¹¹C-labeled brain tracers injected 10-30 minutes apart with a single dynamic PET scan. Though the multi-tracer PET signal was not separated into individual tracer components in this work and images of each tracer were not recovered, it did demonstrate recovery of certain rate parameters from a dual-tracer dataset.

Interest in myocardial perfusion imaging (MPI) with PET/CT is increasing with the widespread availability of PET/CT scanners, tracer distribution networks, and the forthcoming arrival of ¹⁸F-labeled myocardial blood flow tracers. Conventional methods utilize rest and then stress imaging which requires separate scans to be performed at rest and then under either exercise or pharmacologic stress. Using signal-separation strategies similar to those for rapid multi-tracer PET imaging, single scan techniques can be used to acquire a rest image and then a stress cardiac image in a single scan. This brings the benefits of increased throughput, native co-registration of the rest and stress images, and reduced radiation exposure since only one CT scan is needed for attenuation correction, provides natively co-registered rest and stress images, and offers an improved patient experience.

Previous work on single-scan rest/stress MPI PET has focused on a rest-first protocol. Specifically, rest-first imaging generally takes the following steps: position the patient, administer the rest tracer and begin dynamic imaging, induce pharmacologic stress after 5-8 minutes, then administer the stress tracer and continue imaging dynamically. While this work has represented an advancement over previous methods, rest/stress-based imaging methods may present some disadvantages. For example, such methods cannot utilize exercise stress (which has various advantages over pharmacologic stress) without having the patient exit the imaging system, thereby causing difficult image misregistration problems when the patient reenters the imaging system. Additionally, a significant number of patients experience physiological changes when undergoing stress, such as transient left ventricle dilation (TLVD). These changes complicate and limit the usefulness of rest-first methods.

### BRIEF SUMMARY

The present application provides for systems and methods which utilize stress-first and then rest imaging techniques to provide for improved medical imaging results. One embodiment may administer a stress regime, such as exercise stress, and administer a tracer to a patient and retrieve a stress image. A second tracer is administered shortly thereafter to a patient, and a resting image is retrieved. Embodiments may implement this method with PET/CT scanning techniques. Additionally, embodiments may utilize a single CT scan when obtaining both the stress and rest images.

Utilizing stress-first imaging provides advantages which have not heretofore been recognized and would be counterintuitive to one of ordinary skill in the art. For example, the capability of obtaining a rest image after, and in close proximity to, a stress regime has not been previously contemplated. The ability to obtain a rest image in proximity to a stress image allows methods to utilize exercise stress and take a sequence of images without requiring a patient to exit the imaging system and/or make large movements which could frustrate patient registration. Additionally, utilizing stress-first imaging allows embodiments to compensate for physical changes (e.g. TLVD) caused by stress regimes. Further, a stress-first regime may assist in better distributing tracer agents, thereby allowing for lower doses of tracer to be used, which in turn assists in reducing noise in a subsequent resting image.

In accordance with one example embodiment, a method for performing multi-tracer PET cardiac imaging is provided. The method comprises: introducing a first tracer into a subject at a state of cardiac stress, acquiring a first set of PET cardiac imaging data of the subject to obtain a first set of tracer data corresponding to a cardiac stress image, introducing a second tracer into the subject at a state of cardiac rest, acquiring a first set of PET cardiac imaging data of the subject to obtain a second set of tracer data corresponding to a cardiac rest image, providing a kinetic model which estimates time-dependent activity of the first and second tracers, and applying the first and second set of tracer data to the kinetic model to recover images corresponding to the cardiac stress and cardiac rest images.

Another embodiment may be characterized as a medical imaging system which includes a scanning device configured to obtain a first stress-based image of the subject and a subsequent rest-based image of the subject. Further the system includes a processing device configured to apply an image processing model which estimates time-dependent activity of a first and second tracer which was used to obtain the first stress-based image and subsequent rest-based image in order to process out noise in the rest-based image which corresponds to the stress-based image.

Another embodiment provides for a computer program product comprising a non-transitory computer-readable medium comprising code for causing a processor to: receive a first set of medical imaging data corresponding to a scan of a subject which is in a stressed state, store said first set of medical imaging data, receive a second set of medical imaging data corresponding to a scan of a subject which is in a rest state, and process the second set of medical imaging data to compensate for noise due to a tracer which was present during the first scan of a subject in a stressed state in order to create a processed rest image.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings, in which:

FIG. 1A illustrates one embodiment of a computing device that can be used to practice aspects of the preferred embodiment.

FIG. 1B illustrates an alternative embodiment of a processing system that may be used.

FIG. 2A illustrates conventional methods for measuring cardiac blood flow at rest and during stress with a tracer.

FIG. 2B illustrates a previous method for rapid dual-injection, single-scan imaging processes.

FIG. 2C illustrates a method with utilizes a rest-first imaging protocol where the rest tracer is administered prior to imaging, but the rest image and stress image are acquired in a single scan.

FIG. 2D illustrates a method with utilizes a stress-first imaging protocol in accordance with an embodiment of the present application.

FIG. 3 illustrates dual-state compartment models for separable activity distributions and accompanying time-activity curves for a single scan stress-first protocol in accordance with an embodiment of the present application.

FIG. 4 illustrates dual-state compartment models for inseparable activity and accompanying time-activity curves for a single scan stress-first protocol in accordance with an embodiment of the present application.

FIG. 5 illustrates kinetic modeling for a single tracer using principal component analysis (PCA) for estimating and extrapolating the tracer's kinetic behavior in accordance with an embodiment of the present application.

FIG. 6 illustrates myocardial perfusion PET images obtained using the single-scanning session protocols illustrated in FIGS. 2B, 2C, and 2D.

FIGS. 7A-7C show linear regression analysis and scatter plots comparing uncorrected and corrected voxel values in the left ventricle myocardium versus the standard values obtained from conventional separate-scan imaging.

FIGS. 8A-8C show the sum-squared error for image voxels for the three imaging protocols depicted in FIGS. 2B, 2C, and 2D.

FIGS. 9A-9C show contrast for blow flow defects in the left ventricle myocardium for the three imaging protocols depicted in FIGS. 2B, 2C, and 2D.

FIG. 10 illustrates a flowchart implementing a method in accordance with an embodiment of the present application.

### DETAILED DESCRIPTION

Before the present methods and systems are disclosed and described, it is to be understood that this invention is not limited to specific synthetic methods, specific components, or to particular compositions, as such may, of course, vary. For example, specific types of imaging devices, types of imaging targets (e.g. cardiac), etc., are described. However, it is to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The present invention may be understood more readily by reference to the following detailed description of preferred embodiments according to the invention and the Examples included therein and to the Figures and their previous and following description.

Herein, the term “tracer” is used to identify each individual tracer or administration of a tracer, and will be used generically to refer to both tracers of different chemical form and multiple administrations of the same tracer at different times and/or under different physiological conditions (e.g. at rest and stress for myocardial perfusion imaging).

Likewise, the term “multi-tracer” refers to data containing contributions from more than one tracer as defined above (such that rapid sequential rest/stress myocardial perfusion imaging constitutes multi-tracer imaging in that there are two tracer administrations—one at rest and another at stress—wherein a part of the PET data contains signals arising from both tracer administrations).

The term PET “signal” is broadly used to describe the essence of the PET measurement under discussion. To varying degrees multi-tracer PET signal separation can be performed on the raw scanner data, partially processed data, reconstructed dynamic images, and/or time-activity curves; similarly, for each tracer, the imaging endpoint(s) may be a static image, standardized uptake value (SUV), pseudo-quantitative measure, kinetic parameter(s) and/or macro parameter(s). For a given dataset and imaging endpoint, “signal” is used to identify the element or elements of the dataset necessary for computing the desired endpoint. Likewise, “signal separation” (and “signal recovery”) refer to the process of separating a multi-tracer dataset into individual tracer components, thereby recovering the necessary signal for each tracer for computing the desired endpoint.

As will be appreciated by one skilled in the art, the preferred embodiment may be implemented as a method, a data processing system, or a computer program product. Accordingly, the preferred embodiment may take the form of an entirely hardware embodiment, an entirely software embodiment, or an embodiment combining software and hardware aspects. Furthermore, implementations of the preferred embodiment may take the form of a computer program product on a computer-readable storage medium having computer-readable program instructions (e.g., computer software) embodied in the storage medium. More particularly, implementations of the preferred embodiments may take the form of web-implemented computer software. Any suitable computer-readable storage medium may be utilized including hard disks, CD-ROMs, optical storage devices, or magnetic storage devices.

The preferred embodiments according to the present invention are described below with reference to block diagrams and flowchart illustrations of methods, apparatuses (i.e., systems) and computer program products according to an embodiment of the invention. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

### I. Computer or Computing Device

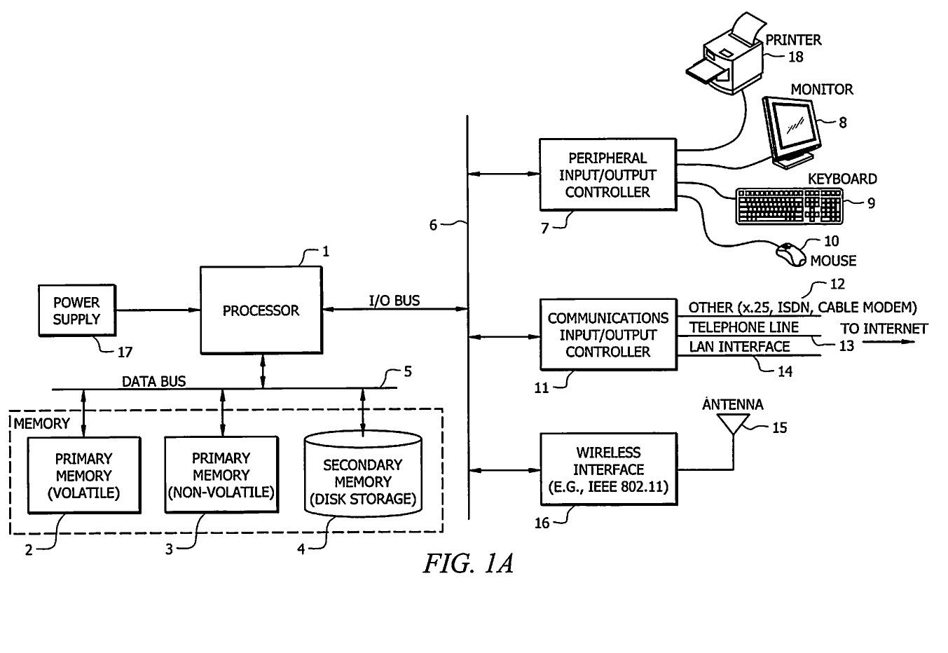

In the embodiments referenced herein, a “computer” or “computing device” may be referenced. Such computer may be, for example, a mainframe, desktop, notebook or laptop, a hand held device such as a data acquisition and storage device, or it may be a processing device embodied within another apparatus such as, for example, a scanner used for tomography. In some instances the computer may be a “dumb” terminal used to access data or processors over a network. Turning to FIG. 1A, one embodiment of a computing device is illustrated that can be used to practice aspects of the preferred embodiment. In FIG. 1A, a processor 1, such as a microprocessor, is used to execute software instructions for carrying out the defined steps. The processor 1 receives power from a power supply 17 that also provides power to the other components as necessary. The processor 1 communicates using a data bus 5 that is typically 16 or 32 bits wide (e.g., in parallel). The data bus 5 is used to convey data and program instructions, typically, between the processor and memory. In the present embodiment, memory can be considered primary memory 2 that is RAM or other forms which retain the contents only during operation, or it may be non-volatile 3, such as ROM, EPROM, EEPROM, FLASH, or other types of memory that retain the memory contents at all times. The memory could also be secondary memory 4, such as disk storage, that stores large amount of data. In some embodiments, the disk storage may communicate with the processor using an I/O bus 6 instead or a dedicated bus (not shown). The secondary memory may be a floppy disk, hard disk, compact disk, DVD, or any other type of mass storage type known to those skilled in the computer arts.

The processor 1 also communicates with various peripherals or external devices using an I/O bus 6. In the present embodiment, a peripheral I/O controller 7 is used to provide standard interfaces, such as RS-232, RS422, DIN, USB, or other interfaces as appropriate to interface various input/output devices. Typical input/output devices include local printers 18, a monitor 8, a keyboard 9, and a mouse 10 or other typical pointing devices (e.g., rollerball, trackpad, joystick, etc.).

The processor 1 typically also communicates using a communications I/O controller 11 with external communication networks, and may use a variety of interfaces such as data communication oriented protocols 12 such as X.25, ISDN, DSL, cable modems, etc. The communications controller 11 may also incorporate a modem (not shown) for interfacing and communicating with a standard telephone line 13. Finally, the communications I/O controller may incorporate an Ethernet interface 14 for communicating over a LAN. Any of these interfaces may be used to access a wide area network such as the Internet, intranets, LANs, or other data communication facilities.

Finally, the processor 1 may communicate with a wireless interface 16 that is operatively connected to an antenna 15 for communicating wirelessly with another device, using for example, one of the IEEE 802.11 protocols, 802.15.4 protocol, or a standard 3G wireless telecommunications protocols, such as CDMA2000 1x EV-DO, GPRS, W-CDMA, or other protocol.

An alternative embodiment of a processing system that may be used is shown in FIG. 1B. In this embodiment, a distributed communication and processing architecture is shown involving a server 20 communicating with either a local client computer 26a or a remote client computer 26b. The server 20 typically comprises a processor 21 that communicates with a database 22, which can be viewed as a form of secondary memory, as well as primary memory 24. The processor also communicates with external devices using an I/O controller 23 that typically interfaces with a LAN 25. The LAN may provide local connectivity to a networked printer 28 and the local client computer 26a. These may be located in the same facility as the server, though not necessarily in the same room. Communication with remote devices typically is accomplished by routing data from the LAN 25 over a communications facility to a wide area network 27, such as the Internet. A remote client computer 26b may execute a web browser, so that the remote client 26b may interact with the server as required by transmitted data through the wide area network 27, over the LAN 25, and to the server 20.

Those skilled in the art of data networking will realize that many other alternatives and architectures are possible and can be used to practice the preferred embodiments. The embodiments illustrated in FIGS. 1A and 1B can be modified in different ways and be within the scope of the present invention as claimed.

### II. Overview

Described herein are embodiments of a method of recovering component stress and rest signals or estimates of component stress and rest signals from combined signals of multiple tracers in the context of imaging multiple PET tracers, a single tracer injected repeatedly, or a combination of tracers using, e.g., static, multiple-timepoint or dynamic scanning, where the tracer administrations are simultaneous or staggered in time such that some or all of the PET timeframes, images, data, and/or datasets contain overlapping signals from more than one of the tracer administrations.

The multi-tracer or multi-state PET imaging signal includes components from all of the tracer administrations. Mathematically this is described by letting R_{dual}(t) represent the PET signal at time t, including contributions from both the stress tracer injection and the rest tracer injection. Since the signals from each individual tracer are not explicitly distinguishable, the multi-tracer or multi-state PET signal is the sum of the stress and rest signals:

R_{dual}(t)=Rₛₜᵣₑₛₛ(t)+Rᵣₑₛₜ(t) (1)

In general, the process of signal separation is to recover Rₛₜᵣₑₛₛ(t) and Rᵣₑₛₜ(t) from R_{dual}(t). In the following discussion, a tilde (˜) is used to indicate that a variable is a (noisy) measured quantity, a bar (−) to indicate it is modeled, and a caret (̂) to indicate that it is estimated or recovered.

A number of algorithms for separating multi-tracer or multi-state PET datasets into individual-tracer or individual-state components can be used in accordance with some embodiments, where the recovered data for each tracer can be subsequently analyzed by conventional single-tracer or single-state methods. These algorithms include background subtraction and model-based signal separation comprised of model-guided signal separation and model-restricted signal separation. Each of the multi-tracer signal separation algorithms utilizes models or analysis methods that describe the dynamic behavior of the tracers administered at either stress or rest; these models may describe radioactive decay of static tracer distributions, dynamically changing tracer distributions, or both. In general these kinetic models are well understood for modeling a single tracer at either rest or stress, but generally have not been applied to multi-state PET data where one tracer administered at a cardiac stressed state and the second is administered at resting state. Without loss of generality, the signal will often be described as a time-activity curve in this discussion since the concepts are generally more easily present in that context. However, it is to be appreciated that single-tracer kinetic analysis methods have been applied in both projection space and image space, and that multi-tracer kinetic models and signal separation algorithms can likewise be applied in the same manners and should not be construed to be limited in application to time-activity curves per se.

The general premise for multi-tracer or multi-state signal separation is that the kinetic behavior of each tracer obeys certain constraints—and when injections at cardiac stress and cardiac rest are separated in time, these constraints provide sufficient information to recover the signal components due to each tracer from the overlapping portions of the time-activity curves. There are a variety of way to perform the signal separation and recover separated and corrected estimates of the stress and rest imaging signals, including Background Subtraction algorithms with a variety of signal extrapolation techniques as well as Model-Based Signal Separation algorithms employing a variety of kinetic models. Examples of these algorithms are described in U.S. Pat. No. 7,848,557, the disclosure of which is incorporated by reference herein. Application of those algorithms to multi-tracer or multi-state stress-rest cardiac imaging involves applying the same general mathematical principals, but to the specific case of a tracer administered during cardiac stress followed by a second tracer administered at a cardiac rest state after the cardiac stress has subsided.

### III. Examples and Evaluation Methods

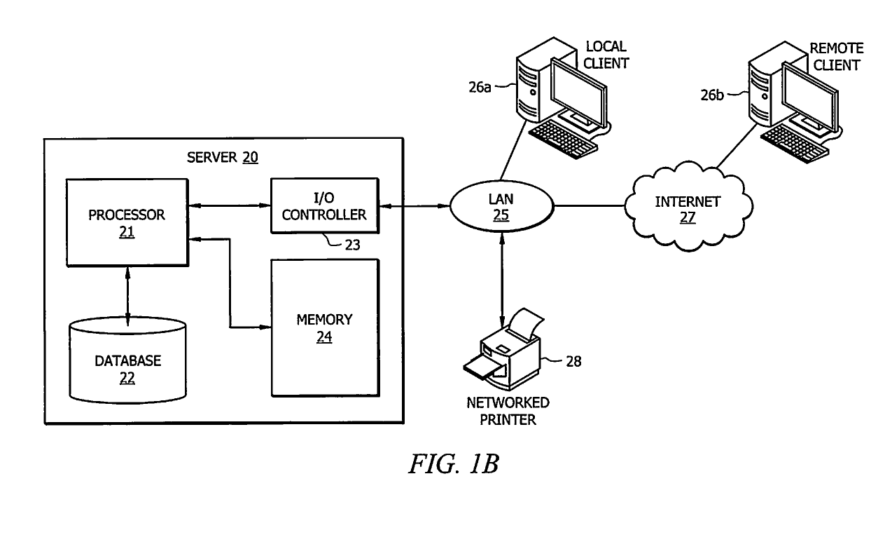

FIG. 2A illustrates conventional methods for measuring cardiac blood flow at rest and during stress with a tracer such as, for example, ¹³N-ammonia or ¹⁸F-flurpiridaz PET requiring a waiting period between scans to allow for radioactive decay. It is noted that this figure is depicted with static imaging at both rest and stress, although dynamic imaging at either or both could optionally be used. A previous method for rapid dual-injection, single-scan imaging processes is illustrated in FIG. 2B. In FIG. 2B, after the patient is positioned in the scanner and a transmission scan has been acquired for attenuation correction, dynamic PET is performed continuously while injections of a tracer such as, for example, ¹³N-ammonia are administered at the scan start during rest and a short time later (e.g., 10 minutes) during adenosine stress. This rapid dual-injection approach reduced the overall procedure time significantly compared to conventional single-injection methods, e.g. increased scanner throughput and utilization, improved co-registration of rest and stress data, reduced motion artifact, reduced transmission scan radiation exposure and improved patient comfort and convenience. Another advantage is that if the two tracer doses are the same (for example, the first injection and the second injections are ¹³N-ammonia), the injections may be obtained from a single cyclotron run and split for the rapid sequential injections. However, as noted above, the previous rest-first protocols provided for several unappreciated disadvantages.

FIG. 2C illustrates another method with utilizes a rest-first imaging protocol. As illustrated, a patient may first be injected with a first tracer (such as ¹³N-ammonia or ¹⁸F-flurpiridaz) at cardiac rest and await a period for tracer uptake and distribution. The patient is then positioned on the imaging table and pharmacologic stress is induced. A single CT scan or transmission scan may be used to provide for attenuation correction of the PET images and anatomic visualization. PET scanning commences to acquire either a static or multi-frame dynamic image of the rest tracer activity. Either during, or proximate to the stress regime, a second tracer (the “stress tracer”, such as ¹³N-ammonia or ¹⁸F-flurpiridaz) is administered. PET scanning continues to acquire an image or multiple images as the stress tracer distributes.

FIG. 2D illustrates a method with utilizes a stress-first imaging protocol in accordance with an embodiment of the present application. As illustrated, a patient may first undergo exercise or pharmacologic stress. Either during, or proximate to the time of administering the stress regime, a first tracer (such as ¹³N-ammonia or ¹⁸F-flurpiridaz) is administered. As described above, in accordance with some embodiments, this first injection may utilize lower doses for a first tracer than previous methods. This is due to the fact that the stress environment provides for better tracer distribution in the body. Because of the lower dosage, the first tracer may have a reduced effect on subsequent images.

When the first tracer is sufficiently distributed, embodiments may utilize a single CT scan to provide for attenuation correction of the PET images and anatomic visualization. The use of a single CT scan is advantageous because it lowers the radiation exposure for the patient over previous methods that may have required a CT scan while taking the separate rest and stress images. The illustrated embodiment then utilizes static PET imaging to obtain the stress image. In this example embodiment, static imaging is utilized as would normally be the case when exercise stress is provided for. However, embodiments may utilize dynamic PET imaging.

Upon receiving the stress image, a second tracer injection is administered. Thereafter, either a static or dynamic PET scan may be utilized. This method is implemented in a single session which does not require a patient to exit the imaging system. Accordingly, improved coregistration between images is obtained. Further, as noted above, these methods also improve coregistration issues which occur due to stress-based physical changes, e.g. TLVD.

### IV. Processing Methods

Example processing methods which utilize dual-state processing algorithms are described herein. Each of the dual-state scanning protocols requires a post-processing correction in order to recover valid and uncorrupted images at both rest and stress. While the particulars of each scanning protocol differ, the same dual-state processing concept applies to each case—estimate the stress and rest components of the PET imaging signal and separate them to recover stress-only and rest-only images. Each protocol gives rise to somewhat different dual-state processing requirements. The requirements for each depends on the extent to which the residual activity from the first injection has fully distributed by the time of the second injection. The relevant algorithms may be classified into two categories: Dual-State Modeling for Separable Activity Distributions, and Dual-State Modeling for Inseparable Activity Distributions.

### V. Dual-State Compartment Modeling for Separable Activity Distributions

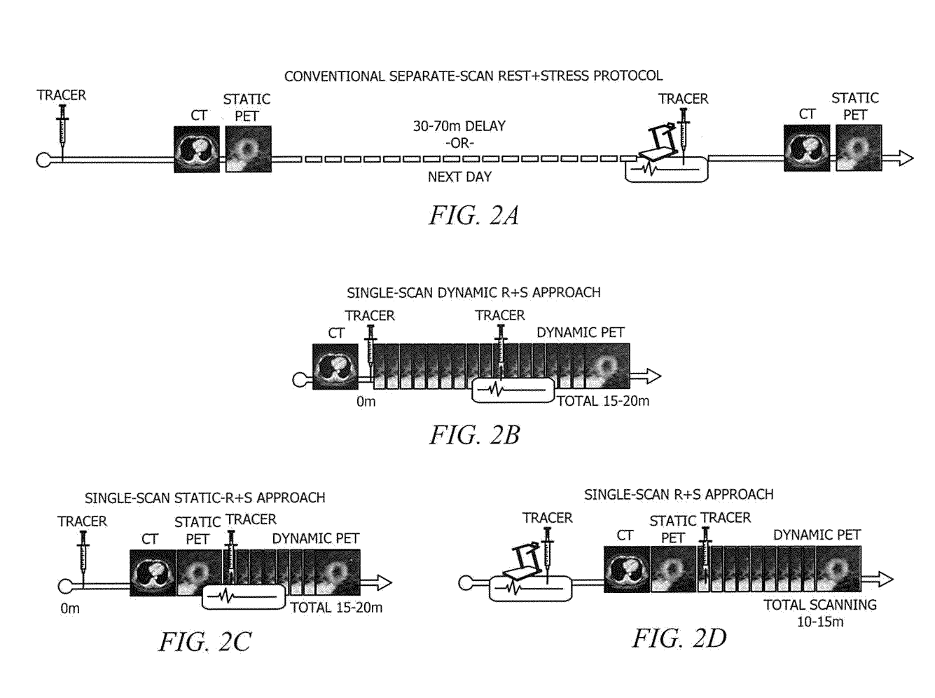

FIG. 3 illustrates dual-state compartment models and accompanying time-activity curves for a single scan stress-first protocol in accordance with an embodiment of the present application. First the patient is stressed, either pharmacologically or via exercise, and tracer is administered at peak stress. After waiting a suitable uptake period for the stress activity to distribute, the patient is positioned on the scanner and a conventional stress scan is acquired. The patient then remains on the scanner, and a dynamic rest scan is acquired along with a second tracer administration. The determination of whether or not the activity distributions can be considered separable or inseparable depends on the activity concentration in the extravascular exchangeable compartment (C₁^{stress}(t) in FIG. 2) at the time that the rest scan is started. If there is no activity in this compartment at this time, then the stress activity is fully distributed, and the dynamic rest scan with second tracer injection provides an entirely new set of activity that can be treated separately from the stress activity. However, if significant activity is present in C₁^{stress}(t) at the time the rest scan is started, then the activity distributions may be classified as inseparable. In most cases with myocardial blood flow tracers that are rapidly extracted and trapped in the myocardium, the separable activity model will be valid within a few minutes after tracer injection.

The dual-state compartment model for separable activity distributions can be written:

$${\hat{R}}^{Dual} ( t < {t}_{stress} ) = {f}_{B}^{rest} B ( t ) + ( 1 - {f}_{B}^{rest} ) {A}^{rest} ( t ) and$$

$${\hat{R}}^{Dual} ( t < {t}_{stress} ) = {f}_{B}^{rest} B ( t ) + ( 1 - {f}_{B}^{rest} ) {A}^{rest} ( t )$$

$${A}^{rest} ( t ) = \frac{{K}_{1}^{rest} {k}_{3}^{rest}}{{k}_{2}^{rest} + {k}_{3}^{rest}} {\int }_{0}^{t} {e}^{- λ ( t - τ )} {b}^{rest} ( τ ) + \frac{{K}_{1}^{rest} {k}_{2}^{rest}}{{k}_{2}^{rest} + {k}_{3}^{rest}} {e}^{- ( {k}_{2}^{rest} + {k}_{3}^{rest} + λ ) t} ⊗ {b}^{rest} ( t ) with {b}^{rest} ( t < {t}_{stress} ) = 0$$

$${A}^{stress} ( t ) = \frac{{K}_{1}^{stress} {k}_{3}^{stress}}{{k}_{2}^{stress} + {k}_{3}^{stress}} {\int }_{0}^{t} {e}^{- λ ( t - τ )} {b}^{stress} ( τ ) + \frac{{K}_{1}^{stress} {k}_{2}^{stress}}{{k}_{2}^{stress} + {k}_{3}^{stress}} {e}^{- ( {k}_{2}^{stress} + {k}_{3}^{stress} + λ ) t} ⊗ {b}^{stress} ( t ) with {b}^{stress} ( t < {t}_{stress} ) = 0$$

Note that the dual-state approach with two tracer injections also affects decay correction, and decay correction cannot be performed prior to separating the rest and stress images (since they include a combination of both tracer injections prior to separation, no single decay correction factor can be used). This is easily solved by incorporating the decay correction (X) into the compartment modeling equations as shown above.

In its simplest form, e.g. when the residual activity has completely distributed prior to the commencement of imaging, dual-state kinetic modeling for separable activity distributions reduces to a simple background subtraction. When the residual activity is incompletely distributed prior to the commencement of imaging but well defined by the imaging period prior to injection of the second tracer, the residual activity distribution can be extrapolated and more complex background subtraction algorithms are effective. When the activity distribution is not yet well defined, however, then dual-state modeling for inseparable activity distributions is warranted. VI. Dual-State Kinetic Modeling for Inseparable Activity Distribution

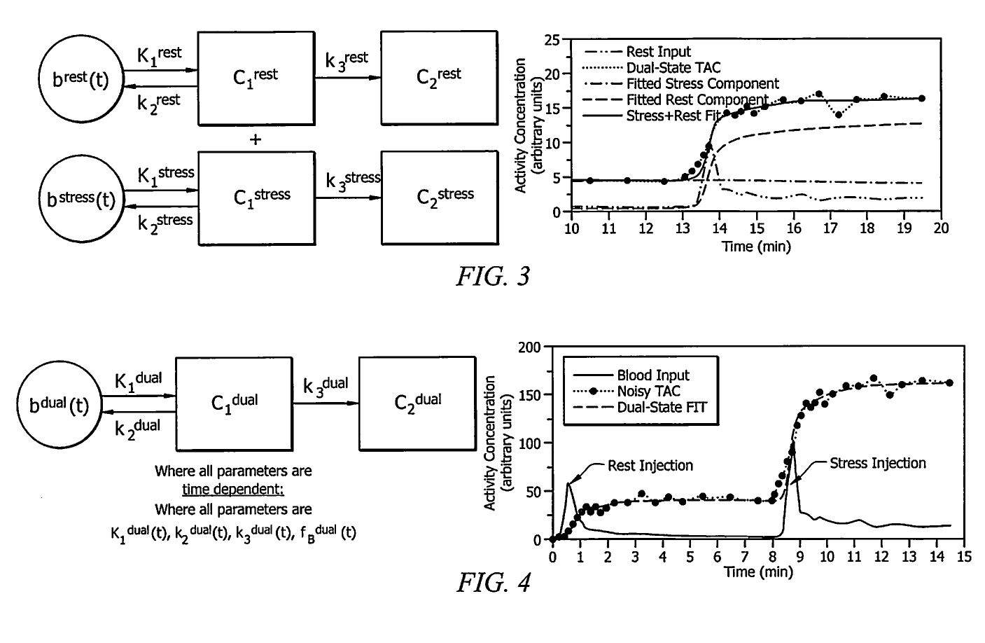

FIG. 4 illustrates dual-state compartment models for inseparable activity and accompanying time-activity curves for a single scan stress-first protocol in accordance with an embodiment of the present application. When the activity distributions from the stress and rest injections cannot be treated as separable (e.g., when significant activity is present in C₁^{rest}(t) when stress is induced), then the dual-state kinetic model for inseparable activity shown in FIG. 4 is preferably used. In this case, a single input function is present, b^{Dual}(t), including activity from both tracer injections; however, all kinetic parameters are time-dependent and vary between rest and stress values over time.

Several versions of this model can be considered, depending on how the kinetic parameters are constrained to change in time. The simplest transition is the instantaneous change model, where each parameter takes on either rest or stress values, instantaneously changing between values when adenosine is infused:

$${A}^{Dual} ( t < {t}_{stress} ) = ⌊ {K}_{1}^{rest} {k}_{3}^{rest} / ( {k}_{2}^{rest} + {k}_{3}^{rest} ) {e}^{- λ t} + {K}_{1}^{rest} {k}_{2}^{rest} / ( {k}_{2}^{rest} + {k}_{3}^{rest} ) {e}^{- ( {k}_{2}^{rest} + {k}_{3}^{rest} + λ ) t} ⌋ ⊗ b ( t )$$

$${A}^{Dual} ( t \geq {t}_{stress} ) = ⌊ {K}_{1}^{stress} {k}_{3}^{stress} / ( {k}_{2}^{stress} + {k}_{3}^{stress} ) {e}^{- λ ( t - {t}_{stress} )} + {K}_{1}^{stress} {k}_{2}^{stress} / ( {k}_{2}^{stress} + {k}_{3}^{stress} ) {e}^{- ( {k}_{2}^{stress} + {k}_{3}^{stress} + λ ) ( t - {t}_{stress} )} ⌋ ⊗ b ( t - {t}_{stress} ) + {C}_{e} ( {t}^{stress} ) [ {k}_{3}^{stress} / ( {k}_{2}^{stress} + {k}_{3}^{stress} ) {e}^{- λ ( t - {t}_{stress} )} + {k}_{2}^{stress} / ( {k}_{2}^{stress} + {k}_{3}^{stress} ) {e}^{- ( {k}_{2}^{stress} + {k}_{3}^{stress} + λ ) ( t - {t}_{stress} )} ] + {C}_{m} ( {t}^{stress} ) {e}^{- λ ( t - {t}_{stress} )}$$

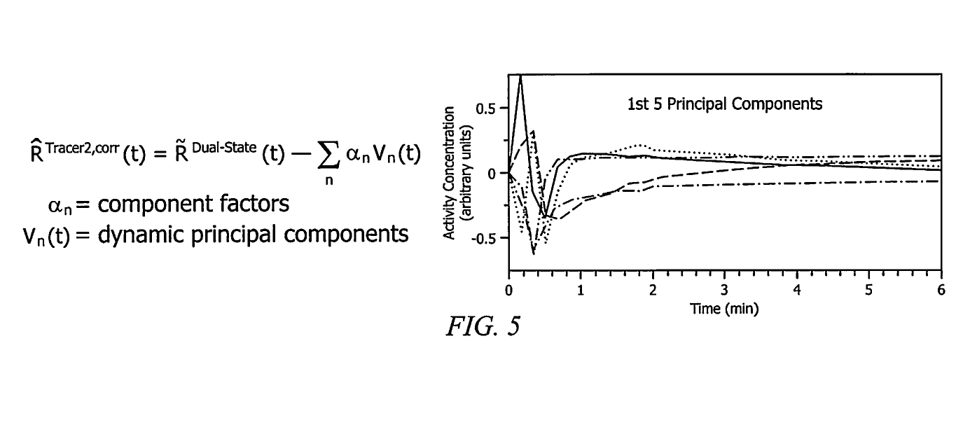

FIG. 5 illustrates kinetic modeling using principal component analysis (PCA) to model and predict the timecourse of activity from one or more tracer administrations. In various implementations, the principal components may be obtained from PCA analysis of the imaging data itself, from a population database of imaging or time-activity curve data, or from single- or dual-state compartment modeling using technique such as those just described. PCA-based kinetic modeling can be used with both separable or inseparable activity distributions. Likewise they can be used to extrapolate residual first tracer activity for Background Subtraction correction algorithms, and they can also be used for dual-state modeling using either Model-Guided or Model-Restricted signal-separation techniques.

### VII. Experimental Results

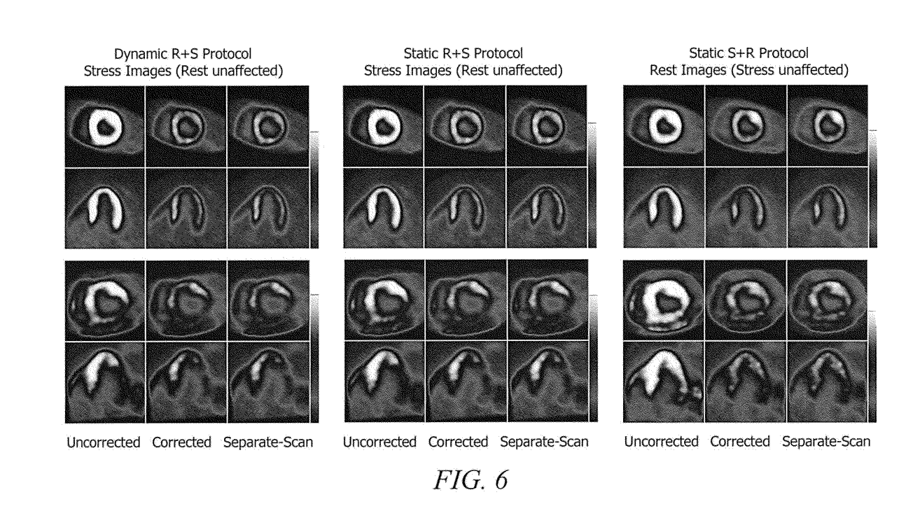

Example experimental data are included herein to illustrate certain embodiments and compare them with previous dynamic rest+stress methods. FIG. 6 shows example PET images of ¹³N-ammonia acquired using the Dynamic R+S protocol (left) in accordance with previous methods; acquired using a Static R+S (center); and Static S+R (right) protocols in accordance with an example embodiment of the present application. The uncorrected images display an excess of tracer activity throughout the myocardium and background. After correction using PCA-based correction methods as outlined above, the corrected images closely match the separate-scan gold standard images. Here the upper set of images show a patient with relatively uniform tracer uptake, whereas the lower set of images show a patient with severe blood flow defects.

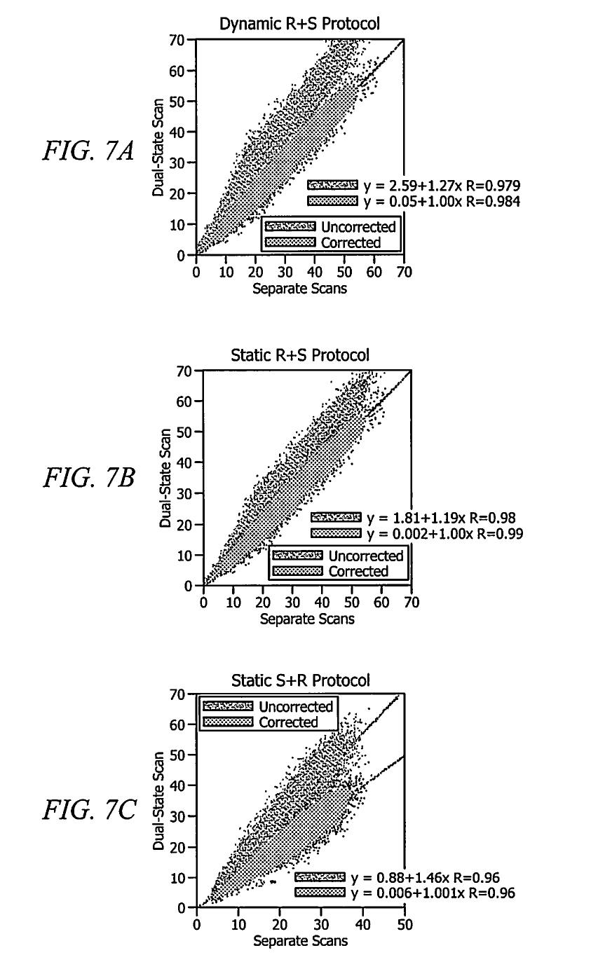

FIGS. 7A-7C show scatter plots and linear regression analysis for the lower set of images from FIG. 6, comparing uncorrected and corrected image voxel values with separate-scan gold standard values. In all three cases the uncorrected values had significant bias (slopes greater than 1.0, non-zero intercepts) due to the presence of residual tracer from the first injection. After correction, the bias in the voxel values was largely removed. In addition, the correlation coefficients demonstrate improved correlations for the corrected data as compared to the uncorrected data.

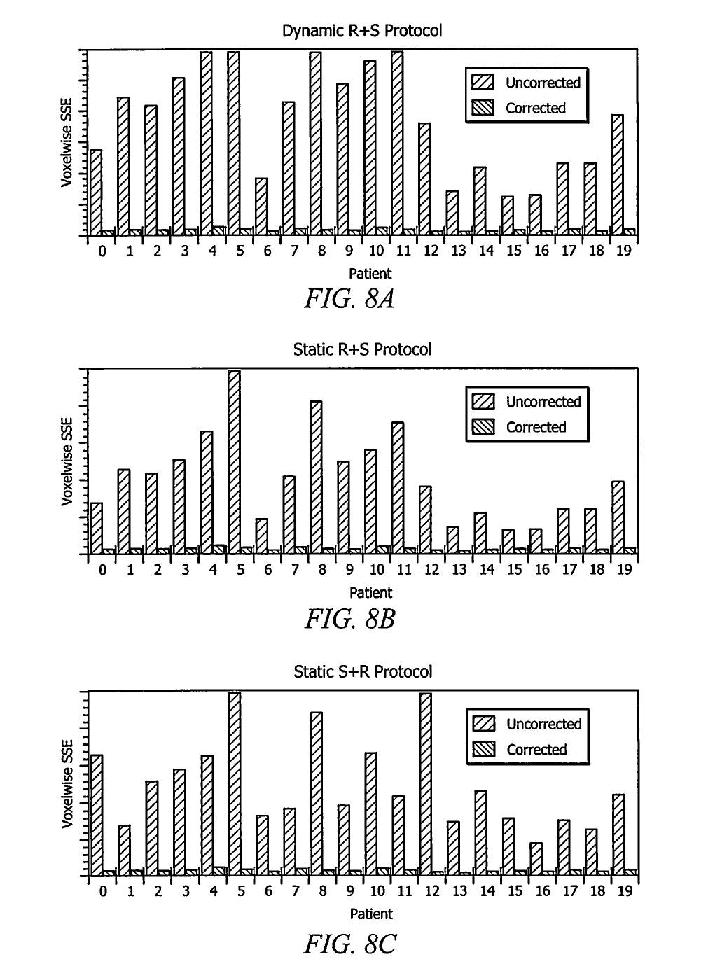

FIGS. 8A-8C show quantitative image analysis results from 19 patients, computing the sum-squared error (SSE) over all image voxels for the uncorrected and corrected images, using conventional separate scan images as standards. Large SSEs were observed for all uncorrected images, and these errors were reduced to near zero for the corrected images.

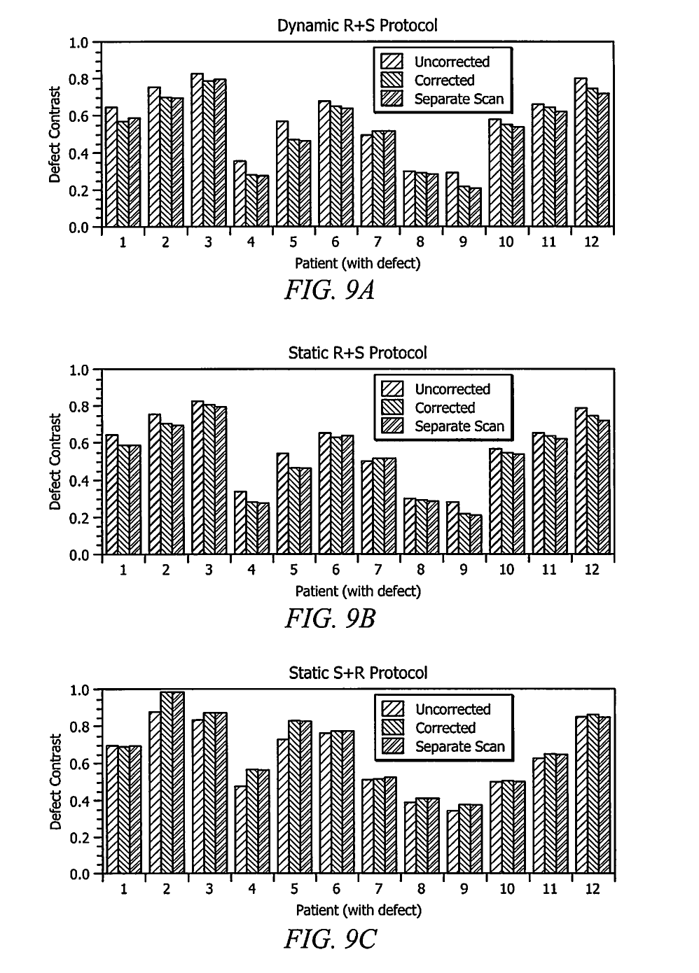

FIGS. 9A-9C show analysis of defect contrast in 12 patients that had left ventricle myocardial blood flow defects. Defect contrast for the uncorrected images differed from defect contrast for the standard images, where the uncorrected defect contrast was too high in some patients and too small in others. These could lead to false positive or false negative results in some cases. After correction, the defect contrasts more closely matched the separate scan gold standard values.

### VIII. Example Implementation Methods

In view of exemplary systems shown and described herein, methodologies that may be implemented in accordance with the disclosed subject matter will be better appreciated with reference to various functional block diagrams. While, for purposes of simplicity of explanation, methodologies are shown and described as a series of acts/blocks, it is to be understood and appreciated that the claimed subject matter is not limited by the number or order of blocks, as some blocks may occur in different orders and/or at substantially the same time with other blocks from what is depicted and described herein. Moreover, not all illustrated blocks may be required to implement methodologies described herein. It is to be appreciated that functionality associated with blocks may be implemented by software, hardware, a combination thereof or any other suitable means (e.g., device, system, process, or component). Additionally, it should be further appreciated that methodologies disclosed throughout this specification are capable of being stored on an article of manufacture to facilitate transporting and transferring such methodologies to various devices. Those skilled in the art will understand and appreciate that a methodology could alternatively be represented as a series of interrelated states or events, such as in a state diagram.

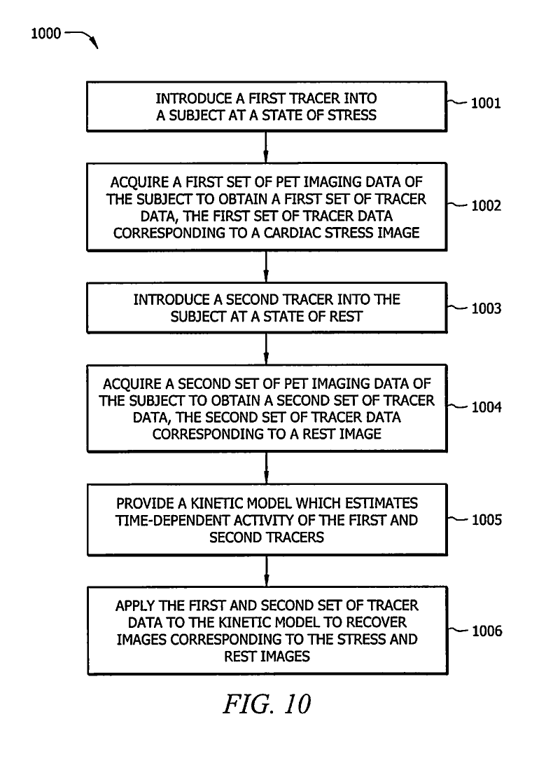

FIG. 10 illustrates an operational flow 1000 for a process which obtains a stress/rest imaging data in accordance with an embodiment of the present application. Method 1000 begins by introducing a first tracer into a subject at a state of stress 1001. The first tracer may consist of any element which is capable of providing for discernible imaging results. Additionally, the first tracer preferably will have a short half-life in order to reduce interference/noise from the first tracer on any subsequent images. Additionally, as stated above, the state of stress may be induced by any means such as through exercise and/or pharmacological. Moreover, stress may be induced by a plurality of means.

While not shown in method 1000, it is noted that once the first tracer has been administered, some embodiments may acquire a CT scan of the subject in order to obtain data for attenuation correction of the PET images. Some embodiments may utilize a single CT scan at this point in time, while others may use multiple scans. It is appreciated that the ability to utilize a single in some embodiments is advantageous over prior methods which would require multiple scans. Specifically, utilizing only one CT scan functions to shorten the amount of time needed for imaging, reduce the amount of radiation exposure, etc.

Method 1000 then performs acquires a first set of PET imaging data of the subject to obtain a first set of tracer data, the first set of tracer data corresponding to a stress image 1002. This first image will ordinarily be obtained using a static PET technique. However, in some embodiments a dynamic imaging method may be used. It is appreciated that a dynamic imaging method would likely utilize pharmacologic stress regimes due to the need for a patient to be stationary.

After receiving the first image, method 1000 then introduces a second tracer into the subject at a state of rest at 1003. The second tracer may be a different element than the first tracer, or may be the same element. It is noted that the second element is not as sensitive to half-life considerations as it will not likely be interfering with subsequent scans. It may be advantageous for the sake of efficiency and cost to utilize an element which has a half-life such that the first and second tracers may be produced concurrently.

When the second tracer is distributed in the subject, method 1000 then acquires a second set of PET imaging data of the subject to obtain a second set of tracer data, the second set of tracer data corresponding to a rest image at 1004. This second image will ordinarily be obtained using a dynamic PET technique, however, static methods may also be used. Embodiments may also be implemented in a manner which minimizes the subjects movements between the first and second image scans. Such a minimization will function to reduce errors due to image misregistration. It is noted that acquiring the first and second sets of PET imaging data may be implemented in a single PET scan, or by using multiple scans which are relatively proximate in time before subject exits (or substantially moves within) the imaging device.

Method 1000 may also include the step of providing a kinetic model which estimates time-dependent activity of the first and second tracers 1005. Such a kinetic model may be designed to simply assist in reducing the effects of the first tracer in the second image.

Further, the kinetic model may also be designed to assist with other factors which may cause image irregularities such as partial volume correction, model-based noise regularization, and the like; further, the kinetic model may also serve to quantify tracer uptake or quantify blood flow. In one embodiment the kinetic model is at least comprised in part of a compartment model for one or more of the first tracer and second tracers. Additionally, some embodiments may apply a background subtraction technique which is used to correct or compensate the cardiac rest second tracer data for the presence of residual cardiac stress first tracer data. Such a subtraction technique may be utilized along with or instead of kinetic modeling techniques.

Finally, method 1000 applies the first and second set of tracer data to the kinetic model to recover images corresponding to the stress and rest images 1006. These finalized images may then be stored on a processing device, transmitted to a third party, etc.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method for performing multi-tracer PET cardiac imaging, the method comprising:
introducing a first tracer into a subject at a state of cardiac stress;
acquiring a second set of PET cardiac imaging data of the subject to obtain a first set of tracer data, said first set of tracer data corresponding to a cardiac stress image;
introducing a second tracer into the subject at a state of cardiac rest;
acquiring a first set of PET cardiac imaging data of the subject to obtain a second set of tracer data, said second set of tracer data corresponding to a cardiac rest image;
providing a kinetic model which estimates time-dependent activity of the first and second tracers; and
applying the first and second set of tracer data to the kinetic model to recover images corresponding to the cardiac stress and cardiac rest images.

2. The method of claim 1 wherein state of cardiac stress has been achieved using exercise-based stress.

3. The method of claim 1 wherein state of cardiac stress has been achieved using pharmacological-based stress.

4. The method of claim 1 further comprising performing a single CT scan of the subject in conjunction with performing the cardiac PET imaging.

5. The method of claim 1 wherein acquiring at least one of said first and second sets of PET cardiac imaging data is implemented as a dynamic scan.

6. The method of claim 1 wherein acquiring at least one of said first and second sets of PET cardiac imaging data is implemented as a static scan.

7. The method of claim 1 wherein said first and second cardiac PET scans are implemented without substantially moving the subject between scans.

8. The method of claim 1 wherein said first and second tracers are the same tracer element.

9. The method of claim 1 wherein said first and second tracers are different tracer elements.

10. The method of claim 1, wherein the kinetic model is at least comprised in part of a compartment model for one or more of said first tracer and said at least second tracer.

11. The method of claim 1, wherein the kinetic model is at least comprised in part of a model for radioactive decay for one or more of said first tracer and said at least second tracer.

12. The method of claim 1, wherein the kinetic model is at least comprised in part of a model for radioactive decay for one or more of said first tracer and said at least second tracer.

13. The method of claim 1, wherein the kinetic model is at least comprised in part of a model based on component methods such as principal component analysis, spectral analysis, or basis function methods for one or more of said first tracer and said at least second tracer.

14. The method of claim 1 further comprising applying a background subtraction technique, wherein said background subtraction technique is used to correct or compensate the cardiac rest second tracer data for the presence of residual cardiac stress first tracer data.

15. The method of claim 1 wherein acquiring said first and second sets of PET cardiac imaging data is implemented in a single scan.

16. The method of claim 1 wherein acquiring said first and second sets of PET cardiac imaging data is implemented in multiple scans.

17. A medical imaging system comprising:
a scanning device configured to obtain a first stress-based image of the subject and a subsequent rest-based image of the subject;
a processing device configured to apply an image processing model which estimates time-dependent activity of a first and second tracer which was used to obtain said first stress-based image and subsequent rest-based image in order to process out noise in the rest-based image which corresponds to the stress-based image.

18. The medical imaging system of claim 17 further comprising a second scanning device configured to scan a subject to provide attenuation correction data.

19. The medical imaging system of claim 18 wherein said first scanning device is a PET scanning machine and said second scanning device is a CT scanning machine.

20. The medical imaging system of claim 17 wherein said images of a subject are cardiac images.

21. The medical imaging system of claim 20 wherein the state of cardiac stress has been achieved using exercise-based stress.

22. The medical imaging system of claim 20 wherein the state of cardiac stress has been achieved using pharmacological-based stress.

23. The medical imaging system of claim 17 wherein at least one of said first and second scans is implemented as a dynamic PET scan.

24. The medical imaging system of claim 17 wherein at least one of said first and second scans is implemented as a static PET scan.

25. The medical imaging system of claim 17 wherein said first and second scans are implemented without substantially moving the subject between scans.

26. The medical imaging system of claim 17 wherein the image processing model is a kinetic model.

27. A computer program product comprising a non-transitory computer-readable medium comprising code for causing a processor to:
receive a first set of medical imaging data corresponding to a scan of a subject which is in a stressed state;
store said first set of medical imaging data;
receive a second set of medical imaging data corresponding to a scan of a subject which is in a rest state; and
process the second set of medical imaging data to compensate for noise due to a tracer which was present during the first scan of a subject in a stressed state in order to create a processed rest image.
